(19) United States
(12) Patent Application Publication
(10) Pub. No.: US 2011/0236993 A1
(43) Pub. Date: Sep. 29, 2011

(54) PANCREATIC CANCER MARKERS

(75) Inventors: David M. Lubman, Ann Arbor, MI (US); Diane M. Simeone, Ann Arbor, MI (US); Chen Li, Ann Arbor, MI (US)

(73) Assignee: THE REGENTS OF THE UNIVERSITY OF MICHIGAN, ANN ARBOR, MI (US)

(21) Appl. No.: 13/060,802

(22) PCT Filed: Sep. 9, 2009
(86) PCT No.: PCT/US09/56329

§ 371 Date: Jun. 15, 2011

Related U.S. Application Data
(60) Provisional application No. 61/095,793, filed on Sep. 10, 2008.

(51) Int. Cl. G01N 33/574

(52) U.S. Cl. 436/501

(57) ABSTRACT

The present invention relates to pancreatic cancer markers. In particular, the present invention provides methods and compositions for the identification of protein glycosylation patterns associated with pancreatic cancer.

### CROSS REFERENCE TO RELATED APPLICATIONS

This invention claims priority to U.S. Provisional Patent Application Ser. No. 61/095,793, filed: Sep. 10, 2008, which is herein incorporated by reference it its entirety.

### GOVERNMENT SUPPORT

This invention was made with government support under grants 1R21CA124441 and R01 CA106402 awarded by the National Cancer Institute and grant RO1GM49500 awarded by the National Institutes of Health. The government has certain rights in the invention.

### FIELD OF THE INVENTION

The present invention relates to pancreatic cancer markers. In particular, the present invention provides methods and compositions for the identification of protein glycosylation patterns associated with pancreatic cancer.

### BACKGROUND OF THE INVENTION

Pancreatic cancer is most frequent adenocarcinoma and has the worst prognosis of all cancers, with a five-year survival rate of <3 percent, accounting for the 4^{th} largest number of cancer deaths in the USA (Jemal et al., CA Cancer J. Clin., 53: 5-26, 2003). Pancreatic cancer occurs with a frequency of around 9 patients per 100,000 individuals making it the 11^{th} most common cancer in the USA. Currently the only curative treatment for pancreatic cancer is surgery, but only ˜10-20% of patients are candidates for surgery at the time of presentation, and of this group, only ˜20% of patients who undergo a curative operation are alive after five years (Yeo et al., Ann. Surg., 226: 248-257, 1997; Hawes et al., Am. J. Gastroenterol., 95: 17-31, 2000).

The horrible prognosis and lack of effective treatments for pancreatic cancer arise from several causes. Pancreatic cancer tends to rapidly invade surrounding structures and undergo early metastatic spreading, such that it is the cancer least likely to be confined to its organ of origin at the time of diagnosis (Greenlee et al., 2001. CA Cancer J. Clin., 51: 15-36, 2001). Finally, pancreatic cancer is highly resistant to both chemo- and radiation therapies (Greenlee et al., supra). Currently the molecular basis for these characteristics of pancreatic cancer is unknown. What are needed are improved methods for the early diagnosis and treatment of pancreatic cancer. What is needed are serum biomarkers for pancreatic cancer.

### SUMMARY OF THE INVENTION

The present invention relates to pancreatic cancer markers. In particular, the present invention provides methods and compositions for the identification of protein glycosylation patterns associated with pancreatic cancer.

For example, in some embodiments, the present invention provides a method of diagnosing pancreatic cancer in a subject, comprising detecting the presence of a cancer marker (e.g., Alpha-1-β glycoprotein or amyloid). In some embodiments, the detecting comprises detecting the presence of a glycosylated cancer marker. In some embodiments, the detecting comprises the step of binding the cancer marker to a cancer marker specific antibody. In some embodiments, the method further comprises the step of contacting the cancer marker with a lectin (e.g., Aleuria aurentia lectin (AAL), Sambucus nigra bark lectin (SNA), or Lens culinaris agglutinin (LCA)). In some embodiments, the lectin is labeled (e.g., with biotin). In some embodiments, the presence of the glycosylated cancer marker is indicative of pancreatic cancer in the subject.

### DESCRIPTION OF THE FIGURES

FIG. 1 shows an outline of the experimental flow of microarray processing and on-target digestion.

FIG. 2a) the quality of the spots is shown in a fluorescent image of a slide with all fourteen blocks hybridized with the same sample; b), c) and d) the intensity of the signals in the slide shown in a was computed and presented in the three charts in the order of A1BG, Amyloid p component and Antithrombin-III.

FIG. 3 shows the saturation curve of a random serum sample on different antibodies.

FIG. 4 shows MALDI-MS spectra generated on the microarray spots of Amyloid p component antibody after on-target digestion; b) incubated with 10× diluted serum; c) incubated with 2× diluted serum.

FIG. 5 shows fluorescent images of antibody microarray probed with different lectins.

FIG. 6 shows a scatter plot in log 2 scale between every pair of technical replicates (a replicate is two distinct points same patient, same antibody, same fasting status and same batch).

FIG. 7 shows ROC curves for the three antibodies alone and A1BG and Amyloid combined.

FIG. 8 shows a scatter plot of sialylation level detected by lectin SNA on A1BG and Amyloid p component.

FIG. 9 shows a boxplot depicting the distribution of the measurements for antibody A1BG.

### DEFINITIONS

To facilitate an understanding of the present invention, a number of terms and phrases are defined below:

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X,Y locations where the X and Y coordinates are defined by the first and second physical properties, respectively, or vice versa. Such displays also include multi-dimensional displays (e.g., three dimensional displays) that include additional physical properties. In some embodiments, displays are generated by “display software.”

As used herein, the term “detection system capable of detecting proteins” refers to any detection apparatus, assay, or system that detects proteins derived from a protein separating apparatus (e.g., proteins in one or more fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, luminescence etc.) of the protein into a signal that can be processed or stored electronically or through similar means (e.g., detected through the use of a photomultiplier tube or similar system).

As used herein, the term “automated sample handling device” refers to any device capable of transporting a sample (e.g., a separated or un-separated protein sample) between components (e.g., separating apparatus) of an automated method or system (e.g., an automated protein characterization system). An automated sample handling device may comprise physical means for transporting sample (e.g., multiple lines of tubing connected to a multi-channel valve). In some embodiments, an automated sample handling device is connected to a centralized control network. In some embodiments, the automated sample handling device is a robotic device.

As used herein, the terms “centralized control system” or “centralized control network” refer to information and equipment management systems (e.g., a computer processor and computer memory) operable linked to multiple devices or apparatus (e.g., automated sample handling devices and separating apparatus). In preferred embodiments, the centralized control network is configured to control the operations or the apparatus an device linked to the network. For example, in some embodiments, the centralized control network controls the operation of multiple chromatography apparatus, the transfer of sample between the apparatus, and the analysis and presentation of data.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “display screen” refers to a screen (e.g., a computer monitor) for the visual display of computer generated images. Images are generally displayed by the display screen as a plurality of pixels.

As used herein, the term “computer system” refers to a system comprising a computer processor, computer memory, and a display screen in operable combination. Computer systems may also include computer software.

As used herein, the term “directly feeding” a protein sample from one apparatus to another apparatus refers to the passage of proteins from the first apparatus to the second apparatus without any intervening processing steps. In such a case, the second apparatus “directly receives” the protein sample from the first apparatus. For example, a protein that is directly fed from a protein separating apparatus to a mass spectrometry apparatus does not undergo any intervening digestion steps (i.e., the protein received by the mass spectrometry apparatus is undigested protein).

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “sample” is used in its broadest sense. In one sense it can refer to a cell lysate. In another sense, it is meant to include a specimen or culture obtained from any source, including biological and environmental samples. Biological samples may be obtained from animals (including humans) and encompass fluids, solids, tissues, and gases. Biological samples include blood products (e.g., plasma and serum), saliva, urine, and the like and includes substances from plants and microorganisms. Environmental samples include environmental material such as surface matter, soil, water, and industrial samples. These examples are not to be construed as limiting the sample types applicable to the present invention.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

### DETAILED DESCRIPTION OF THE INVENTION

The present invention relates to pancreatic cancer markers. In particular, the present invention provides methods and compositions for the identification of protein glycosylation patterns associated with pancreatic cancer.

Pancreatic cancer continues to have a high mortality rate due to detection at a late stage of the disease (Jemal et al., Cancer J Clin 2006, 56, 106-130). In fact, 85% of patients initially present with advanced, non-resectable disease, highlighting the importance of identifying early detection biomarkers. In addition, in a subset of patients, it may be quite difficult to distinguish chronic pancreatitis and pancreatic cancer, necessitating unnecessary surgery in some patients that otherwise might not require it if an adequate biomarker to distinguish these two diseases was available. A serum biomarker test is expected to improve the efficiency of the diagnosis, where the blood contains the unique secretome of the tumor cells. Several serum markers have been investigated for pancreatic cancer. Elevated CA19-9 level has been cited as a potential marker of disease although it generally does not have the specificity or sensitivity for general screening (Mann et al., Eur J Surg Oncol 2000, 26, 474-479; Ferrone et al., J Clin Oncol 2006, 24, 2897-2902; Duffy et al., Ann Clin Biochem 1998, 35 (Pt 3), 364-370; Boeck et al., Oncology 2006, 70, 255-264; Dalgleish et al., Bmj, 2000; 321: 380; Chang et al., Hepatogastroenterology, 2006; 53: 1-4; Kim et al., J Gastroenterol Hepatol, 2004; 19: 182-186). It has been frequently utilized as a marker to monitor a patient's progress after surgery (Riker et al., Surg Oncol 1998; 6:157-69). Other existing biomarkers relate to the inflammation that associates with the tumor and other pancreatic diseases that may be present (Wigmore et al., Int J Oncol 2002; 21: 881-6; Fearon et al., World J Surg 1999; 23:584-8; Dube et al., Nat. Rev. Drug Discov. 2005; 4, 477-488). It should be noted that no individual biomarker has been found to be conclusive at diagnosis to distinguish chronic pancreatitis and pancreatic cancer (Garcea et al., Eur. J. Cancer 2005, 41, 2213-2236; Rustgi et al., Gastroenterology 2005, 129, 1344-1347). There is no study comparing the serum of pancreatic cancer and diabetes which is a widely existing disease in patients at-risk of pancreatic cancer. Discovery of new early detection biomarkers that are specific for pancreatic cancer remains a major challenge.

Post translational modification of the proteome in serum analysis has become an important area in biomarker research (VanMeter et al., Expert Review of Molecular Diagnostics, 2007; 5; 625-633). Of particular interest is the study of glycoproteins where unique protein glycosylation patterns are associated with cancer (An et al., Anal Chem, 2003; 75: 5628-5637; Block et al., Proc Natl Acad Sci USA, 2005; 102: 779-784; Gessner et al., Cancer Lett, 1993; 75: 143-149; Gorelik et al., Cancer Metastasis Rev, 2001; 20: 245-277; Morelle et al., Curr Pharm Des, 2005; 11: 2615-2645; Peracaula et al., Glycobiology, 2003; 13: 457-470; Poland et al., Prostate, 2002; 52: 34-42; Marrero et al., J Hepatol, 2005; 43: 1007-1012; Ahmed et al., Proteomics, 2005; 5: 4625-4636). Glycans are involved in many biological processes including protein-protein interactions, protein folding, immune recognition, cell adhesion and inter-cellular signaling (Bertozzi et al., Chemical glycobiology. Science, 2001; 291: 2357-2364). Alteration of glycan structure and coverage on several major glycoproteins in serum has been shown to contribute to the progression of cancer. In previous work, fucosylated haptoglobin was suggested as a biomarker for early detection of pancreatic cancer (Okuyama et al., Int. J. Cancer 2006; 118, 2803-2808). Also the glycoforms of alpha-1-acid glycoprotein have been found to vary in cancer patients compared to the healthy controls (Lacunza et al., 2007, 23, 4447-4451). These biomarkers can be used to improve the confidence of the diagnosis through identification of disease-related glycan structures by various separation and mass spectrometry techniques (Yang et al., Journal of Chromatography A, 2004, 1-2, 79-88; Drake et al., Molecular & Cellular Proteomics. 2006, 10, 1957-1967; Cho et al., Analytical Chemistry. 2008, 14, 5286-5292; Kyselova et al., Clinical Chemistry, 2008, 7, 1166-1175). In one such study using lectin extraction and mass spec analysis the glycosylated isoforms of alpha-antitrypsin were shown to change in cancer compared to normal samples or pancreatitis (Zhao et al., Journal of Proteome Research. 2006, 7, 1792-1802). Other studies have removed the glycan groups from the glycoprotein content of the cell and used glycan profiling to show distinct differences between cancer and normal samples based on changes in carbohydrate structures in serum, although association with a particular protein is lost (Zhao et al., Journal of Proteome Research, 2007, 3, 1126-1138). In other studies hydrazide columns have been used to extract glycoproteins from serum which were digested and analyzed by LC-MS/MS. In this report glycoproteins associated with cancer were found although the actual glycan structural information was not delineated (Zhang et al., Nature Biotechnology, 2003, 6, 660-666).

Recently, various microarray formats have been utilized for studying glycosylation patterns. In one study examining sera samples from patients with colon and pancreatic cancers, glycoproteins extracted from serum were printed on glass slides and hybridized against various lectins to study changes in the glycan patterns during cancer progression (Zhao et al., Journal of Proteome Research, 2007, 5, 1864-1874; Qiu et al., Journal of Proteome Research, 2008, 7(4), 1693-1703). This method provides a means of studying subtle changes in glycan structure but does not provide a high throughput mode for further validation. Other methods have included the use of glycan arrays where glycans are directly printed on glass slides (Alvarez et al., Glycobiology. 2006, 292-310) or alternatively lectin arrays where lectins are printed on a slide and glycoproteins or whole cells hybridized against them. The lectin array approach has been used to identify differences in glycoprotein surface markers for cancer cells compared to normal cells and between different types and stages of cancer in several studies (Kuno et al., Nature Methods. 2005, 11, 851-856; Chen et al., Journal of Cancer Research and Clinical Oncology. 2008, 8, 851-860). Alternatively an antibody array approach has been used to capture proteins from serum and a lectin hybridized against the glycoprotein to study changes in glycan structure (Chen et al., Nature Methods. 2007, 5, 437-444). This method can screen large numbers of samples from serum for such changes but requires a discovery platform to choose the antibodies on the array for screening.

The antibody microarray is a favorable format for high throughput analysis, with a high level of specificity and reproducibility (Borrebaec, Expert Review of Molecular Diagnostics, 2007, 7, 673; Ingvarsson et al., Proteomics. 2008, 11, 2211-2219; Haab et al., Current Opinion in Biotechnology. 2006, 4, 415-421; Orchekowski et al., Cancer Research. 2005, 23, 11193-11202). In experiments conducted during the course of development of embodiments of the present invention, antibodies to potential glycoprotein biomarkers were printed on nitrocellulose coated glass slides. The glycans on the printed antibodies were first blocked to eliminate their interference in the hybridization with lectins. The target proteins in the serum were then captured on the antibody array and probed with several biotinylated lectins where streptavidinylated fluorescent dyes were used for detection. Ninety two samples from normal controls, 41 chronic pancreatitis samples, 37 diabetics samples and 22 pancreatic cancer samples were processed using this method where non-cancer samples were randomly selected and all cancer sample available were used. Antibody specificity was verified by on-target digestion of the captured glycoproteins with subsequent on-slide MALDI-MS identification. The data was subjected to statistical analysis to display the variation for a single patient and the differentiation among the disease groups.

Experiments conducted during the course of development of embodiments of the present invention resulted in a antibody/glycoprotein/lectin sandwich assay for screening potential markers of pancreatic cancer. These markers were chosen for study based upon previous work using a lectin glycoarray approach. Three markers were chosen and their corresponding antibodies were printed on coated glass slides. They were exposed to sera from 92 normal samples, 41 chronic pancreatitis samples, 37 diabetic samples and 22 pancreatic cancer samples. The captured glycoproteins were analyzed against four different lectins where SNA was found to provide the best results.

Further, MALDI QIT-TOF MS was used for direct analysis of the captured glycoproteins to optimize dilution conditions of the serum and for minimizing nonspecific binding. It was shown that the pancreatic cancer samples could be clearly distinguished from other disease states and normal samples. The ROC curves showed that Alpha-1-β glycoprotein response to SNA resulted in specific detection of pancreatic cancer with high sensitivity and specificity. The resulting scatterplots also showed the ability to clearly distinguish pancreatic cancer from chronic pancreatitis, diabetics or normal samples. The protein Amyloid also showed the ability to discriminate pancreatic cancer according to the ROC curve whereas Antithrombin-III could not provide such discrimination. A combined ROC curve of Alpha-1-β glycoprotein and Amyloid did not provide any improvement in discrimination due to correlation between the two markers. Additional experiments (e.g., Example 2) demonstrated that the detection methods were able to identify early stage pancreatic cancer.

Accordingly, in some embodiments, the present invention provides systems, kits, and methods for identifying the presence of serum markers indicative of pancreatic cancer. In some embodiments, markers are identified based on their glycosylation patterns (e.g., as described in the Experimental section below).

The present invention is not limited to a particular detection method. In some embodiments, serum proteins (e.g., Alpha-1-β glycoprotein and Amyloid) are identified by first binding to an antibody (e.g., an antibody affixed to a solid support). Specific surface glycosylation patterns are then identified using lectins specific for a particular glycans. In some embodiments, lectins are labeled (e.g., with a fluorescent, chemical or other label) to facilitate detection.

In other embodiments, the presence of glycosylated proteins or protein glycosylation patterns is detected using standard protein detection methods (e.g., those described above). In other embodiments, differences in glycosylation patterns are detected using glycosylation specific methods. For example, in some embodiments, the mass spectrometry methods described herein are utilized to analyze the glycosylation pattern of a specific cancer marker protein. In other embodiments, glycosylation specific reagents (e.g., including, but not limited to, biotinylated or otherwise labeled lectins, glycosylation specific antibodies, or periodic acid-schiff detection methods) are utilized. Reagents for such assays are commercially available.

In some embodiments, a computer-based analysis program is used to translate the raw data generated by the detection assay (e.g., the presence, absence, or amount of a given marker or markers) into data of predictive value for a clinician (See e.g., the above description of data analysis and distribution methods).

In yet other embodiments, the present invention provides kits for the detection and characterization of pancreatic cancer. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In still further embodiments, the kits contain reagents for identifying glycosylated protein (e.g., the glycosylation detection reagents described above). In some embodiments, the kits contain all of the components necessary, sufficient or useful to perform a detection assay, including all controls, directions for performing assays, and any necessary or desired software for analysis and presentation of results.

The compositions and methods of the present invention find use in a variety of research and diagnostic applications. For example, in some embodiments, the kits and methods described herein are utilized in the diagnosis of pancreatic cancer. For example, in some embodiments, individual (e.g., those at increased risk of developing pancreatic cancer) are screened on a regular (e.g., annually or more or less often) basis for the presence of markers indicative of pancreatic cancer (e.g., Alpha-1-13 glycoprotein or Amyloid).

### EXPERIMENTAL

The following examples serve to illustrate certain preferred embodiments and aspects of the present invention and are not to be construed as limiting the scope thereof.

### Example 1

### Experimental

### Sera

Inclusion criteria for the study included patients with a confirmed diagnosis of pancreatic cancer, chronic pancreatitis, long-term (for 10 or more years) Type II diabetes mellitus, or healthy adults with the ability to provide written, informed consent, and provide 40 ml of blood. Exclusion criteria included inability to provide informed consent, patients' actively undergoing chemotherapy or radiation therapy for pancreatic cancer, and patients with other malignancies diagnosed or treated within the last 5 years. The sera samples were obtained from patients with a confirmed diagnosis of pancreatic adenocarcinoma who were seen in the Multidisciplinary Pancreatic Tumor Clinic at the University of Michigan Comprehensive Cancer Center. All cancer sera samples used in this study were obtained from patients with stages III/IV pancreatic cancer. The mean age of the tumor group was 65.4 years (range 54-74 years). The sera from the normal, pancreatitis, and diabetes groups was age and sex-matched to the tumor group. The chronic pancreatitis group was sampled when there were no symptoms of acute flare of their disease. All sera were processed using identical procedures. The samples were permitted to sit at room temperature for a minimum of 30 minutes (and a maximum of 60 minutes) to allow the clot to form in the red top tubes, and then centrifuged at 1,300×g at 4° C. for 20 minutes. The serum was removed, transferred to polypropylene, capped tubes in 1 ml aliquots, and frozen. The frozen samples were stored at −70° C. until assayed. All serum samples were labeled with a unique identifier to protect the confidentiality of the patient. None of the samples were thawed more than twice before analysis. This study was approved by the Institutional Review Board for the University of Michigan Medical School.

### Microarray Preparation and Serum Hybridization

Alpha-1-β glycoprotein antibody was purchased from Novus, while Amyloid p component antibody and Antithrombin antibody were from Abcam. Antibodies were diluted to 50 μg/mL in PBS and spotted on ultra-thin nitrocellulose coated slides (PATH slides, GenTel Bioscience, Madison, Wis.) with a piezoelectric non-contact printer (Nano plotter; GESIM). Each spotting event that resulted in 500 pL of sample being deposited was programmed to occur 5 times/spot to ensure that 2.5 nL was being spotted per sample. The spots used by the MALDI-MS experiment were printed 50 times. Each antibody was printed in triplicate. The spot diameters were 280 um and 700 um for the spots that were printed 5 times and 50 times respectively. The spacing between the spots was 0.7 mm. 14 blocks were printed on each slide in a 2×7 format and the block distance was 9.4 mm.

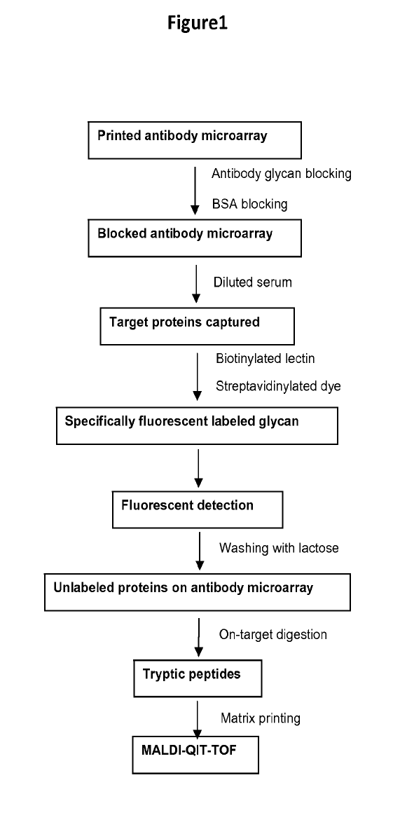

FIG. 1 presents an experimental flow chart of the microarray processing and on-target digestion for MALDI-MS. The antibody arrays on the slides were first chemically derivatized with a method similar to previous work (Peracaula et al., Glycobiology, 2003; 13: 457-470) but modified for this work. The printed slides were dried in an oven at 30° C. for 1 h before gently being washed with PBST 0.1 (100% PBS with 0.1% tween 20) and coupling buffer (0.02M sodium acetate, pH 5.5), and then oxidized by 200 mM NaIO₄ (Sigma) solution at 4° C. in the dark. After 3 hours the slides were removed from the oxidizing solution and rinsed with coupling buffer. The slides were immersed in 1 mM 4-(4-N-maleimidophenyl) butyric acid hydrazide hydrochloride (MPBH; Pierce Biotechnology) at room temperature for 2 hours to derivatize the carbonyl groups. 1 mM Cys-Gly dipeptide (Sigma) was incubated with the antibodies on the slides at 4° C. overnight. The slides were blocked with 1% BSA for 1 hour and dried by centrifugation.

The slides were inserted into the SIMplex (Gentel) 16 Multi-Array device which separates the blocks and prevents cross contamination when different samples are applied on neighboring wells. Serum samples were diluted 10 times with PBST 0.1 containing 0.1% Brij. 100 μL of each sample was applied to the antibody array manually and left in a humidified chamber for 1 hour to prevent evaporation. Slides were rinsed with PBST 0.1 for 3 times to remove unbound proteins. The arrays were then treated with different detection biotinylated lectins (Vector Laboratory) to determine lectin response and streptavidinylated fluorescent dye (Alexa555; Invitrogen Biotechnology) was used for detection. After a final wash, the slides were dried and scanned with a microarray scanner (Genepix 4000A; Axon). The program Genepix Pro 6.0 was used to extract the numerical data. A threshold of signal to background ratio was set at 10 and less than 1% of the spots were under this threshold and excluded. The mean of the intensity in each spot was taken as a single data point into analysis.

### On-Target Digestion and MALDI-QIT-TOF

The microarray slides were incubated with 0.5 M lactose for 10 min and washed with PBST 0.1 to remove the captured lectin from the glycoprotein. After an additional wash with water the slides were dried with centrifugation. Trypsin was diluted to 50 ng/μL with 50 mM ammonium bicarbonate and printed on the microarray spots. The printed slides were moved into a humidity chamber and incubated at 37° C. for 5 min. Thirty five mg/mL 2,5-dihydroxybenzoic acid (DHB) (LaserBio Labs, France) in 50% acetonitrile was printed on the microarray by the microarray printer and allowed to dry.

Mass spectrometric analysis of the microarray slides was performed using the Axima quadrupole ion trap-TOF (MALDI-QIT) (Shimadzu Biotech, Manchester, UK).

The microarray slide was analyzed directly by taping the slide onto the stainless steel MALDI plate and inserting it into the instrument for analysis. Acquisition and data processing were controlled by Launchpad software (Kratos, Manchester, UK). A pulsed N2 laser light (337 nm) with a pulse rate of 5 Hz was used for ionization. Each profile resulted from 2 laser shots. Argon was used as the collision gas for CID and helium was used for cooling the trapped ions. TOF was externally calibrated using 500 fmol/μL of bradykinin fragment 1-7 (757.40 m/z), angiotensin II (1046.54 m/z), P14R (1533.86 m/z), and ACTH (2465.20 m/z) (Sigma-Aldrich). The mass accuracy of the measurement under these conditions was 50 ppm.

### Results and Discussion

### Microarray Printing and Processing

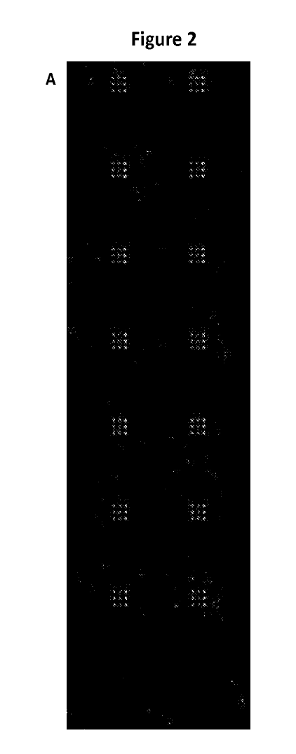

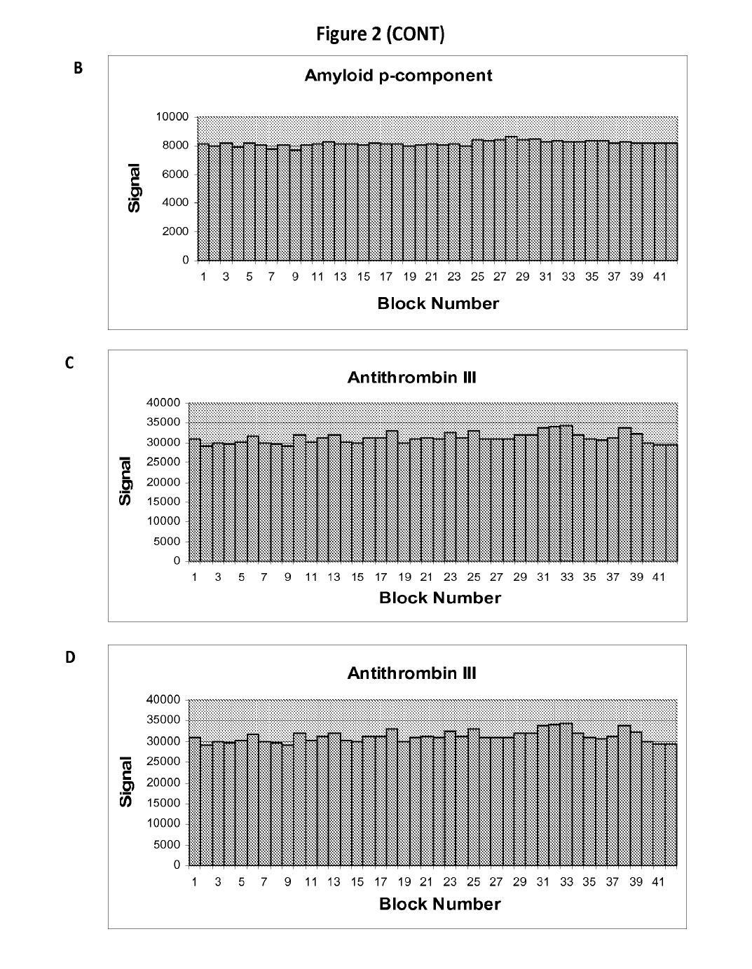

The antibodies were printed on ultrathin nitrocellulose slides and hybridized with serum in a 14 multi-array device, then visualized with biotinylated lectin and Alexafluor-555. In a reproducibility test, a common sample selected at random was applied to all 14 arrays. FIG. 2a illustrates the quality of the printed spots and the variation of the signal over the slides. The intensity of the signal in every single block was analyzed as shown in FIG. 2b. The standard deviation of the signal of any individual antibody within the slides was about 5% of the average. In order to normalize the signal on different slides, 2 blocks on each slide were hybridized with the same two samples. The signals of these two blocks were compared across slides to calculate the normalization ratio. Experiments using multiple slides showed that the slide to slide variation was about 10% of the average signal.

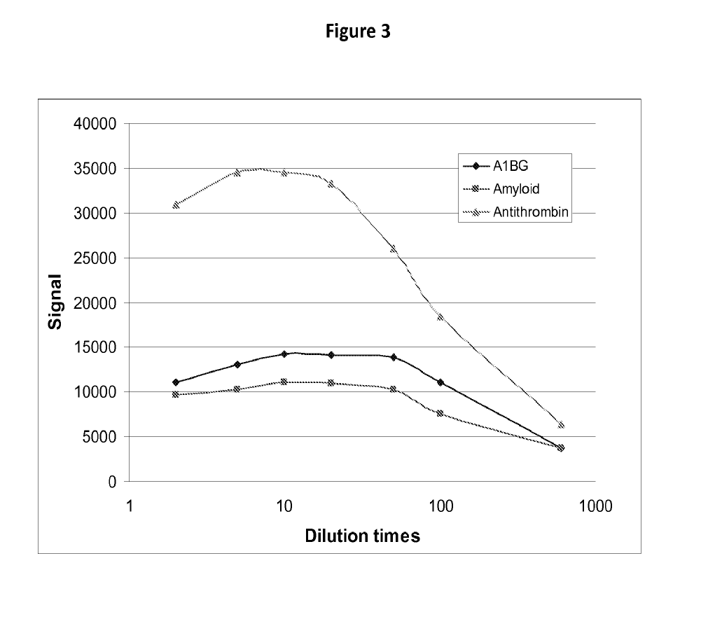

Different dilutions of serum were tested to determine the optimum concentration of the target glycoproteins. There were seven dilutions of serum sample from 2 to 600 times dilution that were applied to the arrays. FIG. 3 depicts the relation between the signal and the fold dilution. A rising trend was noted from the 600× dilution to the 50× dilution for the three glycoproteins shown. In the 50× dilution to the 20× dilution the signal was relatively unchanged except for Antithrombin-III, where the signal increased 20% from the 50× dilution to the 20×. The signal remained the same from the 20× dilution until it reached the 5× dilution, where a saturation of the signal has occurred. A decrease of signal for all three glycoproteins from the 5× dilution to the 2× dilution of serum sample can be seen in the FIG. 3, due to competing non-specific binding on the antibodies.

The result of the dilution test demonstrated that the antibodies were saturated by their target protein at 20× dilution or above in the process of hybridization (1 hour, room temperature and gentle shaking) Below 50× dilution the antibodies were not completely occupied, so the signal decreased with additional dilution. The nonlinear relationship between the concentration of the serum and the intensity of the signal can be attributed to various factors that affect the antibody-antigen reaction, including accessibility of the antibodies, diffusion rate and solubility of the antigen in the hybridization buffer. Nonspecific binding on the antibodies was further investigated and excluded by on-target digestion and MALDI-MS analysis.

To analyze the difference of the glycosylation on potential biomarker proteins, protein expression levels were normalized. The protein level was estimated by antibody assay. In the experiment the three biomarkers were all relatively high abundance proteins in human serum (concentration>20 mg/L) which could easily saturate the antibodies printed on the microarray. Under saturation conditions, the amount of target biomarkers captured on the antibody spots was equal to the capacity of the printed antibody which should be the same in all the replicate blocks. As a result, the need for protein assay was avoided and the intensity of the signal on the microarray directly represented the level of glycosylation.

Antibody Specificity Test with MALDI-QIT-TOF

In order to validate the specificity of the antibodies, on-target digestion and MALDI-QIT-TOF of the spots was performed after elution of biotinylated lectins captured on the glycoproteins with a concentrated sugar solution. A trypsin solution with 50 mM ammonium bicarbonate was printed with the microarray printer using the same spot lay-out as in the antibody printing. The volume of the trypsin solution was 4 nL which in a humidity chamber lasts about 5 minutes before drying out. Ammonium bicarbonate usually decomposes at the same time. 2,5-dihydroxybenzoic acid was then dissolved in 50% acetonitrile and printed on the digested spots. The matrix solution itself is very acidic and stops the digestion to prevent further digestion of antibodies and trypsin autolysis. Acetonitrile also partially dissolved the nitrocellulose film and the digested peptides on the film were extracted and mixed with matrix. Nitrocellulose film has been reported as a excellent substrate for MALDI-MS (Liang et al., Analytical Chemistry, 1998, 3, 498-503). The presence of nitrocellulose in the mixture did not affect the crystallization of DHB.

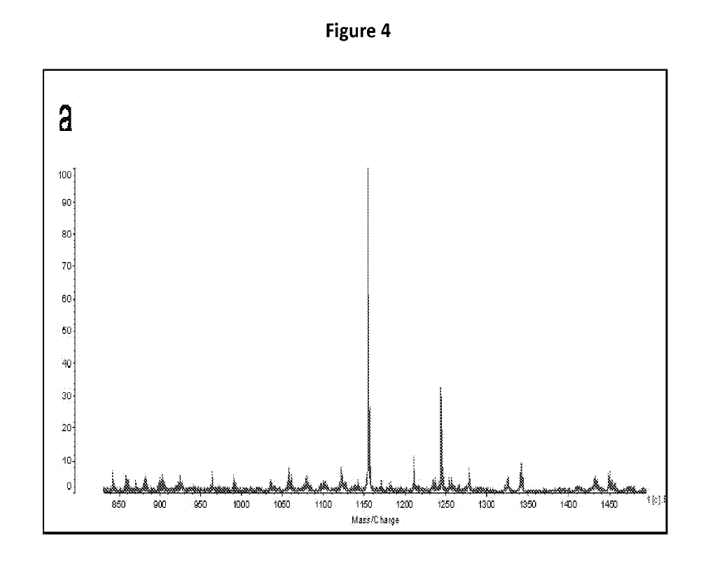

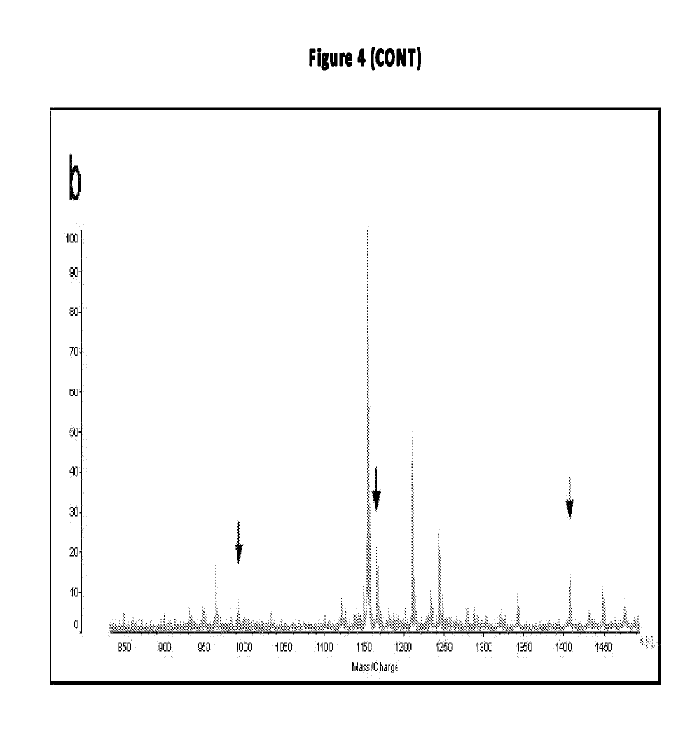

The specificity (specific binding vs. non-specific binding) of the antibody as a function of the dilution times of the serum can be determined by comparing the spectrum from the arrays processed with different conditions. In the experiment one control array (incubated with blocking buffer) and two sample arrays which were hybridized with 2× and 10× dilution of the same serum were tested. The presented figures are the spectra of Amyloid p component antibody spot. FIG. 4a shows the spectrum of the Amyloid p component spot in the control array which only contained the antibody (anti human Amyloid p component). All the peaks in the spectrum are the peptides digested from the antibody and the enzyme itself. The top 3 peaks are attributed to the antibody digest.

The intensity of the other peaks was too low to be identified. The spectra in FIGS. 4b and 4c are generated from the Amyloid p component spots in the sample arrays. In the mass spectrum of 10× dilution, 3 new peaks appeared which were all were identified by MS/MS to be tryptic peptides of Amyloid p component. This result indicated that no other protein was captured on the antibody or the amount was too low to be detected. In the case of the 2× dilution, 2 additional peaks emerged in the spectrum where one of them was identified as human serum albumin while the other one was not identified. The extra peaks are a sign of nonspecific binding on the antibody. Thus, only when the concentration of the sample was increased to 2× the dilution of the serum does non-specific binding begin to affect the specificity of the antibody.

### Detecting Glycosylation on Captured Protein by Blocked Antibody Arrays

The chemical derivatization method was employed to block the glycans on the antibodies to eliminate their binding with the lectins used for detection of glycoproteins (Chen et al., Nature Methods. 2007, 5, 437-444). The cis-diol groups on the glycans were gently oxidized and converted to aldehyde groups which were then reacted with hydrazide-maleimide bifunctional cross-linking reagent and capped with a Cys-Gly dipeptide. After the derivatization reaction the lectins could not recognize the modified oligosaccharide group.

All the antibodies were tested against several samples and lectins to evaluate the effectiveness of the protocol. The underivatized antibodies responded to some of the lectins, but after derivatization the binding greatly decreased or disappeared. The serum solution was incubated against the derivatized antibody array where the spots showed lectin binding on proteins captured by the antibodies, indicating that the antibodies maintained their function after derivatization.

Characterizing Glycan Structure of Potential Biomarkers with Different Lectins

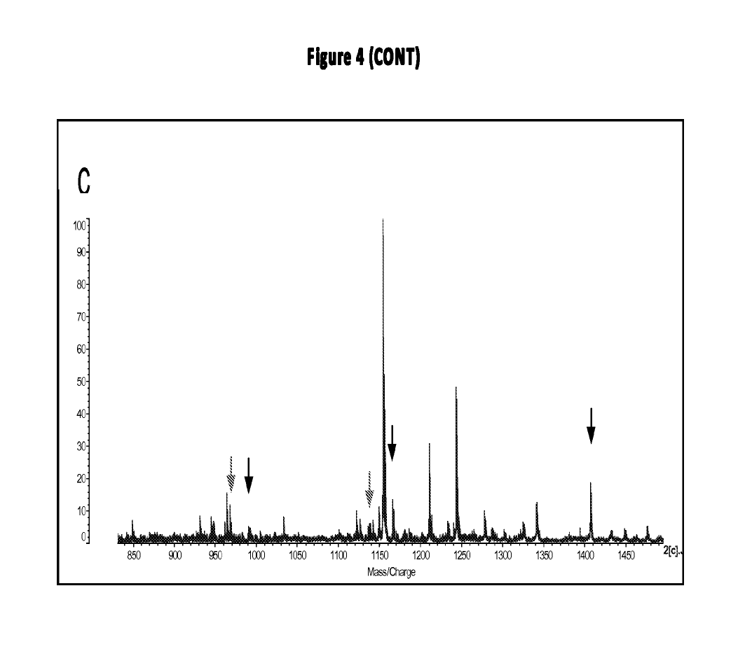

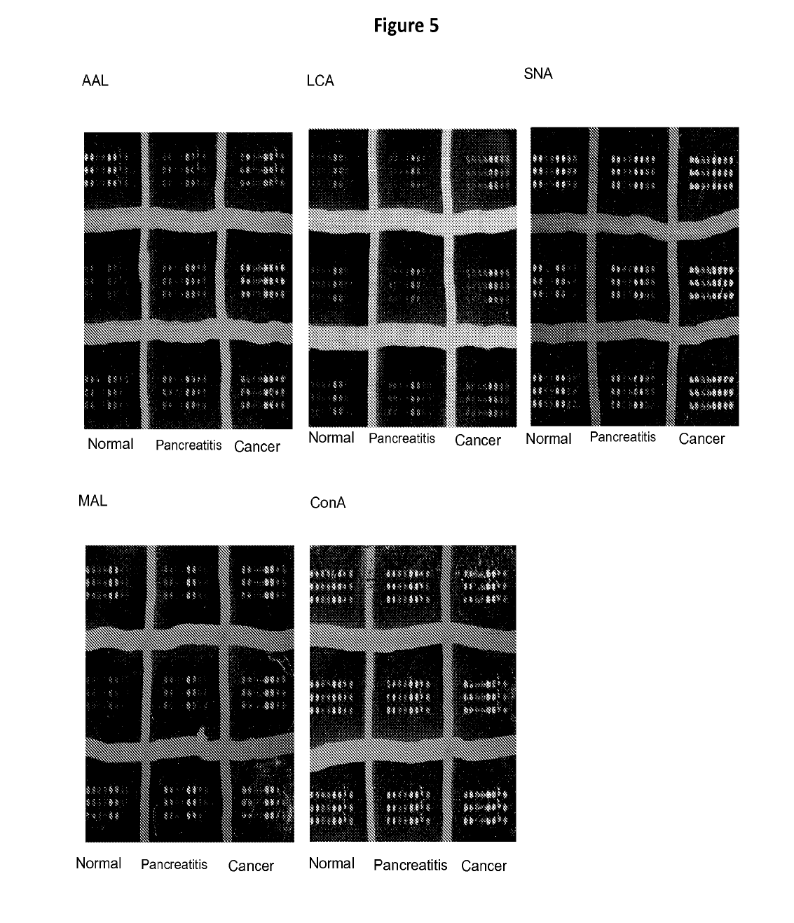

A previous study described ten potential biomarkers in the sera of normal and liver cancer patients that significantly changed their response to several lectins (Ressom et al., 2008, 7, 603-610). Four of these target proteins (Antithrombin-III, Amyloid p component, alpha-1-β glycoprotein and kininogen) were chosen as a proof of concept to determine the proteins which provided the best discrimination of samples from patients in different groups based on lectin response. The biotinylated lectins used were Aleuria aurentia lectin (AAL), Sambucus nigra bark lectin (SNA), Maackia amurensis lectin II (MAL), Lens culinaris agglutinin (LCA), and Concanavalin A (ConA). AAL and LCA bind fucose linked to N-acetylglucosamine or to N-acetyllactosamine related structures. Both MAL and SNA recognize sialic acid on the terminal branches. MAL detects glycans containing NeuAc-Gal-GlcNAc with sialic acid at the 3 position of galactose while SNA binds preferentially to sialic acid attached to terminal galactose. ConA recognizes mannose including high-mannose-type and hybrid-type structures. These lectins were selected since fucosylation and sialylation have been shown to be related to cancer development (Okuyama et al., Int. J. Cancer 2006; 118, 2803-2808; Zhao et al., Journal of Proteome Research. 2006, 7, 1792-1802) and ConA binds to almost all the N-linked glycoproteins where its signal translates into a general level of glycosylation. FIG. 5 shows the result of an initial test of four antibodies and five lectins. The contrast and brightness were optimized to differentiate the three groups. The borders were drawn by hydrophobic marker pens to prevent the cross contamination between the blocks. Three random samples from each group of patients were used. For LCA, AAL, SNA and MAL the three cancer samples all showed a stronger response than the pancreatitis and normal samples, whereas the blocks probed with ConA showed equal signal in the three groups. A binding pattern was shared between LCA and AAL, which agreed with their same specificity on fucosylated N-linked glycans, though the signal of LCA was lower in intensity. These lectins were found to preferentially distinguish normal and chronic pancreatitis samples from cancer samples. MAL was not used for subsequent analysis due to its low sensitivity with these antibodies. Of the 4 antibodies, 3 of them (A1BG, Amyloid p component and Antithrombin-III) displayed a signal-to-background ratio of higher than 20, and were chosen for large set analysis.

### High Throughput Analysis and Data Quality Test

192 samples from patients with various genders, fasting status and disease classes were processed in 4 batches on 16 slides. Since the signal to background ratio for all the valid spots were higher than 10, the signals were directly used for analysis without taking into account the background. 39 of the patients in the groups of normal, chronic pancreatitis and diabetics contributed three samples with two samples collected twice under fasting conditions and the other sample was collected under non-fasting conditions. Two patients provided only double fasting samples which are used for the data quality test. For the other samples including some of the normal, pancreatitis and all the cancer patients, the information of the gender and fasting status is not available. After adjusting for fasting status, gender, and disease category, the data points were compared to a normal reference distribution. Based on this comparison, two outlying data points from the antibody of Antithrombin-III were excluded from all subsequent analysis based on the normal distribution.

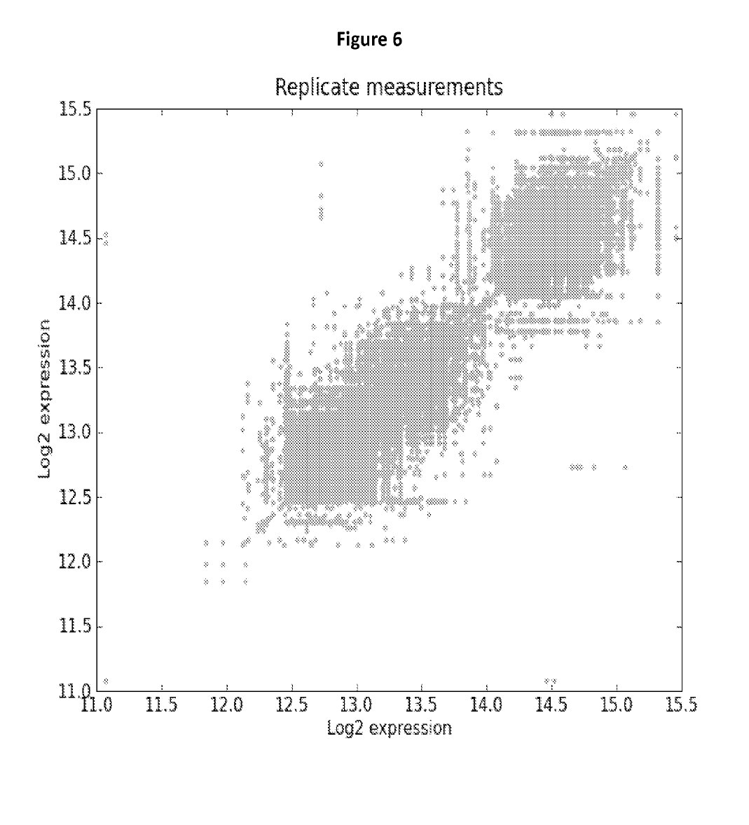

The accuracy of the antibody microarray analysis is heavily dependent on the reproducibility of the technique which is also used as a means to filter out unreliable antibodies in distinguishing cancer from other disease classes. Reproducibility is assessed by fitting a linear mixed effects model to log 2 scale expression data, separately for each antibody. Fixed effects for fasting status, gender, and disease category are included along with random effects for patients, and batches within patients. Thus the expression variation for every antibody around the mean for its fasting/gender/disease group is described in terms of three variance components (residual, patient and batch within patient). Residual variance represents variation for technical replicates (same person, batch, and fasting status). Batch variance represents technical variation for the same person and fasting status across batches. Patient variance represents stable biological variation across people. Table 1 shows the three variance components on the standard deviation scale, for the three antibodies. For example, the residual SD for A1BG is 0.21, which means that two thirds of the replicates will lie within (2̂0.21−1)×100%=16% of the true values and 95% of the replicates will lie within (2̂0.42−1)×100%=34% of the true value. Alternatively, the reproducibility could be exhibited by the correlation of the replicate spots in log 2 scale which is presented in FIG. 6. The scatterplots demonstrate that the technical error is not limited to a handful of outliers, consistent with the finding of an approximately normal distribution of residual variance, as discussed above. FIG. 6 shows data for all non-cancer patients and antibodies pooled.

### Examination of Potential Bias

The present invention is not limited to a particular mechanism. Indeed, an understanding of the mechanism is not necessary to practice the present invention. Nonetheless, it is contemplated that sex, fasting status and other related diseases are all possible sources of bias in biomarker validation (Ransohoff, Nat Rev Cancer 2005; 5:142-9). As discussed above, linear mixed effects models were built separately for each of the three antibodies, with these potentially biasing factors modeled as fixed effects. As always, one level of each factor variable is omitted, so the implicit fixed effect for a normal, non-fasting female is zero, and all other factor settings are interpreted as deviations from this arbitrary baseline setting. The results are listed in Table 2. For A1BG the factors have small and non-significant effects. For Amyloid there is a significant effect for fasting, and for Antithrombin-III there is a significant effect for sex and disease (mainly due to pancreatitis). These effects are statistically significant but are small in magnitude relative to the residual and patient variation, and to the response in cancer.

### Antibody Performance in Distinguishing Cancer and Non-Cancers

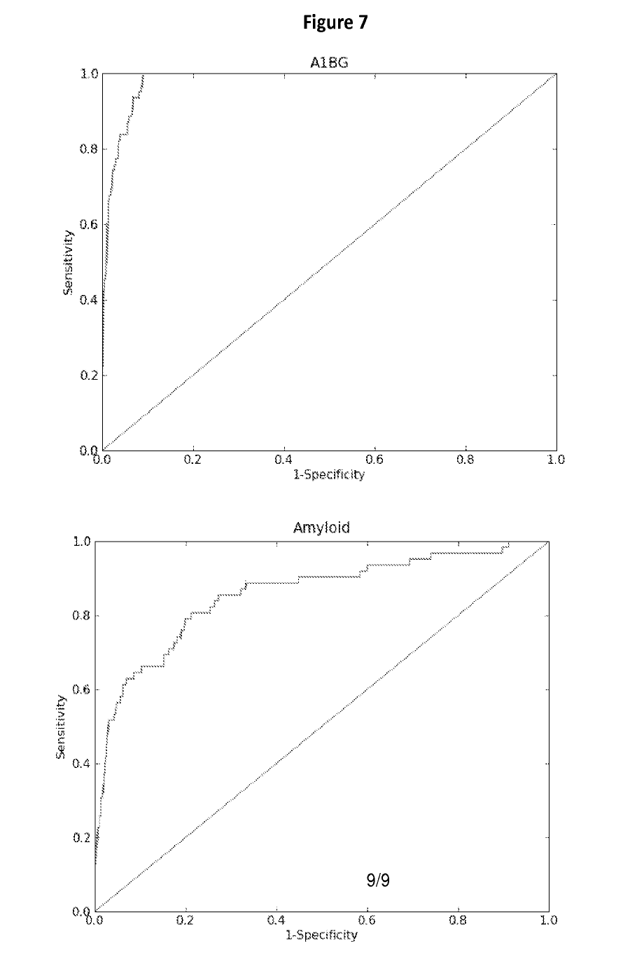

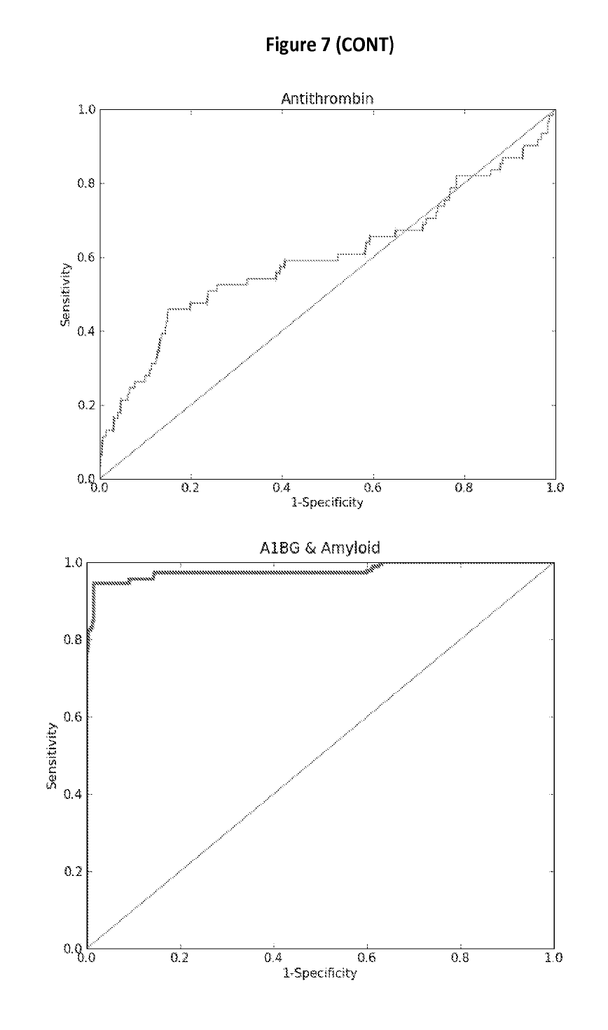

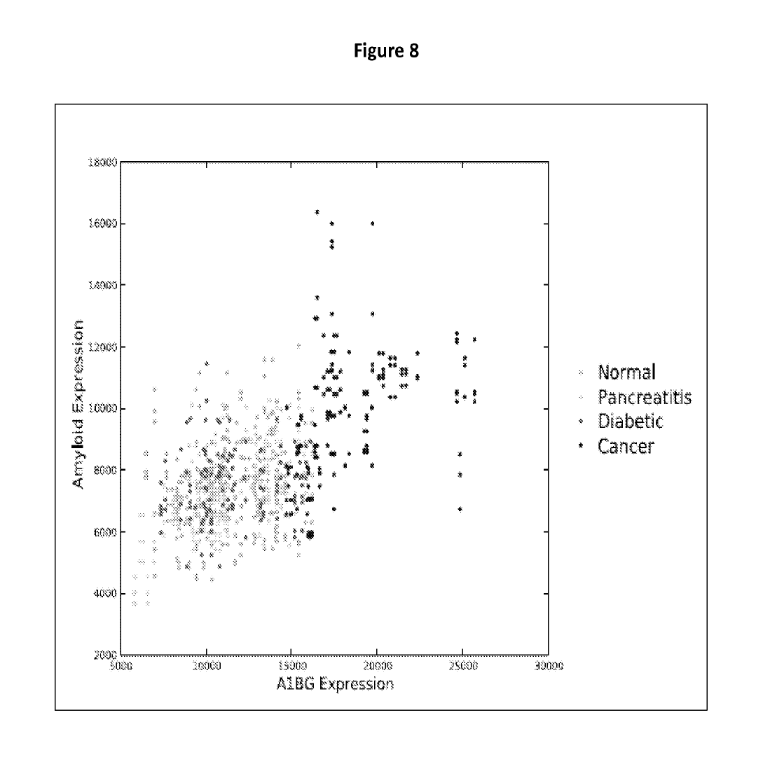

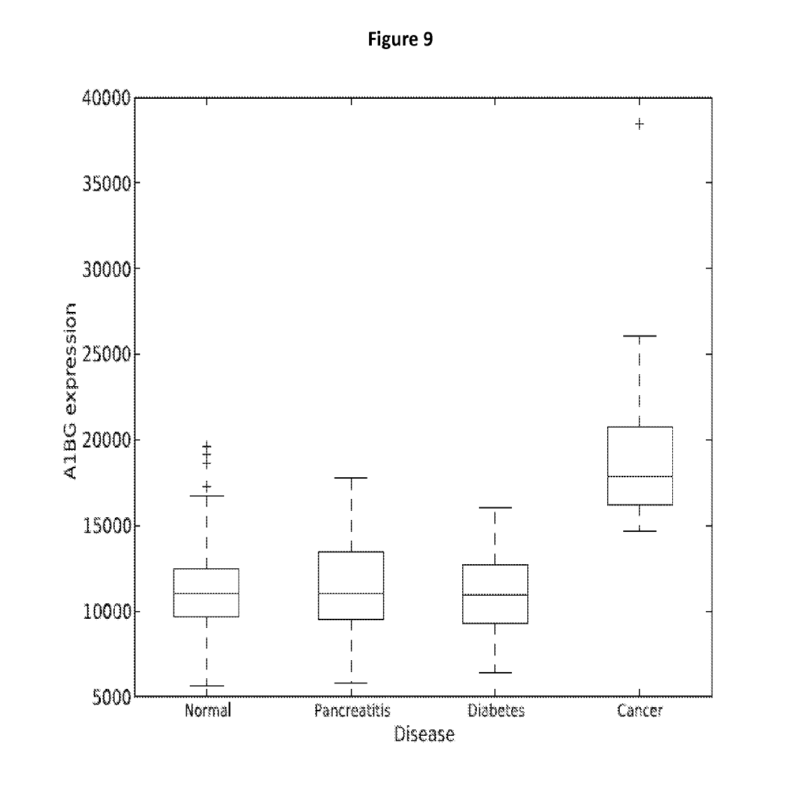

Table 3 provides information concerning the discrimination between cancer and non-cancer samples. The A1BG signal increases by 69% in cancer samples compared to normal, chronic pancreatitis, and diabetic samples. The Amyloid signal increases 33%, and Antithrombin-III is essentially unchanged. The standard deviation from technical and biological variation (within disease classes) is around 0.32 for A1BG. Thus the effect for A1BG is >2 SD where the effect for Amyloid is between 1 and 2 SD. ROC curves in FIG. 7 were also constructed for each of the three markers, based on their ability to distinguish pancreatic cancer from non-cancer samples (a pool of normals, pancreatitis, and diabetes). All three markers show some discrimination where only A1BG is potentially useful on its own. A1BG distinguished cancer and non-cancer samples with a 100% sensitivity and a 98% specificity. The AUC value measuring the area under the ROC curve for A1BG is 0.998. For Amyloid p component the cancer samples were distinguished from non-cancer samples with a 88% sensitivity and a 68% specificity and its AUC value is 0.875. The discrimination for Antithrombin-III is due to the differences between cancer and pancreatitis and it would be unable to distinguish cancer from diabetes based on these data. According to the scatter plot in FIG. 8 where the signals of A1BG and Amyloid were used as X and Y axes, the overlap of the cancer samples with the non-cancer groups is around 20%. The extent of the difference in 4 patient groups is also shown in FIG. 9 which depicts the distribution of the measurement for the antibody A1BG. The boxplot provides the upper and lower quartiles of the measurements with respect to the median value (red line in the middle of each box). The lines provide the ranges of the measurements, excluding outliers (+).

The use of the antibody microarray to capture potential biomarkers available in cancer serum provides a means for high throughput and analysis of glycosylation patterns. Because of the specific goal of quantifying the glycans in this study, antibodies were saturated with the analytes by optimizing the dilution times of the sera according to the saturation curve. Thus the response of the lectin from the microarray directly represented the level of the particular glycosylation without concern about the various concentrations of the proteins in different samples. This strategy also defined the sensitive steps in the experiment where the serum was aliquoted, diluted and hybridized with the microarray, while in other applications of antibody microarrays, factors such as precipitation, heterogeneity of the serum and conditions in hybridization may vary and lead to bias in the method.

Antibody specificity was confirmed by direct MALDI-MS of the microarray spots. Traditional immunoblotting is based on the same interaction as in the antibody microarray and does not exclude undesirable binding. MALDI-MS can identify the tryptic peptides of any captured abundant protein on the target. The microarray printer was essential in precisely depositing the extremely small amount of enzyme and matrix on top of the antibody spots (Evans-Nguyen K M, Tao S C, Zhu H, et al. Protein arrays on patterned porous gold substrates interrogated with mass spectrometry: Detection of peptides in plasma. Analytical Chemistry. 2008, 5, 1448-1458). In this experiment, the nitrocellulose surface generated high quality mass spectra. In spite of peaks from the antibody that dominated the mass spectra, target proteins were readily identified and non-specific binding was also found when the serum was not sufficiently diluted.

To access the technical error of the assay, a comprehensive reproducibility test was applied by using two fasting samples from the same patients (drawn at two times) as pure technical replicates. The samples were disordered before being incubated on the antibody arrays. In most other duplicate studies (Borrebaec, Expert Review of Molecular Diagnostics, 2007, 7, 673; Ingvarsson et al., Proteomics. 2008, 11, 2211-2219; Haab et al., Current Opinion in Biotechnology. 2006, 4, 415-421; Orchekowski et al., Cancer Research. 2005, 23, 11193-11202), variations from an entire slide or batch was more likely to be detected while the individual variability of the single blocks within the slide and batch were ignored. In this work, by statistically comparing the pairs of technical replicates that were distributed across the slides, the divergence of the signal from the ideal value that resulted from the technical error was calculated.

The pancreatic cancer samples could be clearly distinguished from other disease states and normal samples. The ROC curves showed that Alpha-1-β glycoprotein response to SNA resulted in specific detection of pancreatic cancer with high sensitivity and specificity. A combined ROC curve of Alpha-1-β glycoprotein and amyloid did not provide any improvement in discrimination.

TABLE 1

| Std. Dev. (log2) | A1BG | Amyloid | Antithrombin-III |
|---|---|---|---|
| Residual | 0.21559 | 0.19667 | 0.22877 |
| Batch | 0.20701 | 0.16376 | 0.15382 |
| Patient | 0.12681 | 0.05973 | 0.19776 |

TABLE 2

| | Estimate | Pct | Std. Error | T value |
|---|---|---|---|---|
| A1BG | | | | |
| Male | 0.2948 | 2% | 0.05324 | 0.55 |
| Pancreatitis | −0.03688 | −3% | 0.65551 | −0.56 |
| Diabetic | −0.3033 | −2% | 0.06616 | −0.46 |
| Fasting | 0.06761 | 5% | 0.03157 | 2.14 |
| Amyloid | | | | |
| Male | 0.04110 | 3% | 0.03808 | 1.1 |
| Pancreatitis | −0.03545 | −2% | 0.04829 | −0.7 |
| Diabetic | −0.00562 | 0% | 0.04719 | −0.1 |
| Fasting | 0.06383 | 4% | 0.02758 | 2.3 |
| Antithrombin-III | | | | |
| Male | 0.1134 | 8% | 0.04489 | 2.5 |
| Pancreatitis | −0.16112 | −12% | 0.0564 | −2.9 |
| Diabetic | 0.03839 | 3% | 0.05640 | 0.7 |
| Fasting | 0.01663 | 1% | 0.03088 | 0.5 |

TABLE 3

| | Estimate | Pct | Std. Error | T value |
|---|---|---|---|---|
| A1BG | | | | |
| Cancer | 0.75515 | 76% | 0.06972 | 10.8 |
| Pancreatitis | 0.02404 | −2% | 0.06323 | −0.4 |
| Diabetic | −0.01072 | −1% | 0.07169 | −0.1 |
| Amyloid | | | | |
| Cancer | 0.41395 | 33% | 0.06065 | 6.8 |
| Pancreatitis | −0.04436 | −3% | 0.05510 | −0.8 |
| Diabetic | 0.00274 | 0% | 0.06065 | 0.0439 |
| Antithrombin-III | | | | |
| Cancer | 0.03193 | 2% | 0.07523 | 0.4 |
| Pancreatitis | −0.1629 | −12% | 0.06808 | −2.4 |
| Diabetic | 0.02831 | 2% | 0.07523 | 0.4 |

### Example 2

This Example describes detection of early stage pancreatic cancer. The methods described in Example 1 were utilized. The samples showed a glycosylation pattern similar to that of cancer samples. Details of the samples are shown in Table 4.

TABLE 4
Early stage diagnosis

| Research # | Amount | Diagnosis | Stage |
|---|---|---|---|
| 61216931 | 100 μl | Resectable Adenoncarcinoma | T3 N1 MX |
| | | of the Pancreas | |
| 61216931 | 100 μl | Resectable Adenoncarcinoma | T3 N1 MX |
| | | of the Pancreas | |
| 61218761 | 100 μl | Resectable Adenoncarcinoma | T1 NX MX |
| | | of the Pancreas | |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method of diagnosing pancreatic cancer in a subject, comprising detecting the presence of a cancer marker selected from the group consisting of Alpha-1-β glycoprotein and amyloid.

2. The method of claim 1, wherein said detecting comprises detecting the presence of a glycosylated cancer marker.

3. The method of claim 2, wherein said detecting comprises the step of binding said cancer marker to a cancer marker specific antibody.

4. The method of claim 3, further comprising the step of contacting said cancer marker with a lectin.

5. The method of claim 3, wherein said lectin is selected from the group consisting of Aleuria aurentia lectin (AAL), Sambucus nigra bark lectin (SNA), and Lens culinaris agglutinin (LCA).

6. The method of claim 5, wherein said lectin is labeled.

7. The method of claim 6, wherein said label is biotin.

8. The method of claim 2, wherein the presence of said glycosylated cancer marker is indicative of pancreatic cancer in said subject.
